(19) United States
(12) Patent Application Publication
(10) Pub. No.: US 2007/0250118 A1
(43) Pub. Date: Oct. 25, 2007

(54) Compression tissue repair apparatus and methods

(76) Inventor: Michael A. Masini, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/407,775

(22) Filed: Apr. 20, 2006

(51) Int. Cl. A61B 17/08

(52) U.S. Cl. 606/220

(57) ABSTRACT

Instruments and methods facilitate expeditious minimally invasive surgical procedures. A tissue fastener is provided with a female portion and a male portion with a head and a shaft configured to engage with the female portion. A first elongated member is used to hold the female portion of the fastener behind tissue to be repaired. A second elongated member is used to hold the male portion of the fastener in front of tissue to be repaired in alignment with the female portion. The second elongated member is slideable relative to the first elongated member, allowing a user to pierce and compress the tissue as the male portion engages with the female portion. In the preferred embodiment, the male and female portions of the fastener are constructed of a bioabsorbable material such as polylactic acid. The shaft of the male fastener is preferably ribbed or barbed, the female portion of the fastener includes an aperture with features that engage with the ribs or barbs of the male portion, thereby facilitating a desired degree of compression as the portions engage. The head of the male fastener may also be angled for conformance with the outer surface of the tissue undergoing the repair.

### FIELD OF THE INVENTION

This invention relates generally to meniscal repair and, in particular, to instruments and methods directed to expeditious minimally invasive surgical procedures.

### BACKGROUND OF THE INVENTION

FIG. 1 is a simplified drawing of a knee joint. The femur is indicated at 102, the tibia at 103, the patella at 104, and the fibula at 120. The lateral and medial collateral ligaments are shown at 106, 108, respectively. The anterior and posterior cruciate ligaments are depicted at 110, 112.

The meniscus is a horseshoe-shaped piece of cartilage situated between the weight bearing joint surfaces of the knee. It is triangular in cross section and is attached to the lining of the knee joint along its periphery. There are two menisci; the lateral meniscus 114 and the medial meniscus 116. The front third is referred to as the anterior horn, the back third the posterior horn, and the middle third the body. The menisci play an important role in protecting joint cartilage from impact loads. They also cup the joint surfaces of the femur and provide some degree of stabilization to the knee.

There are two different mechanisms for tearing a meniscus: traumatic and degenerative. Traumatic tears result from a sudden impact applied to the meniscal tissue. These usually occur from a twisting injury or a blow to the side of the knee. Degenerative meniscal tears a drying-out of the tissue that progresses with age. The meniscus becomes less elastic and compliant and as a result may fail with only minimal trauma.

A meniscus can tear in almost any conceivable geometric pattern and in any location. Tears confined to the anterior horn of the cartilage however are unusual. Typically tears begin in the posterior horn and then can extend forward into the middle body, as shown in FIG. 1B. A torn meniscus will usually cause pain on the side of the knee proximate to the joint line. Swelling of the joint may also occur.

Since the menisci are largely avascular, a torn meniscus does not have the ability to heal itself, with the exception of small tears confined to the peripheral vascular zone. Surgery is the only way to treat the tear since there are currently no medications, braces, or physical therapy treatments that have been shown to promote healing, at least in the avascular regions.

Based on the location and geometry of the tear the decision is made to either remove (meniscectomy) or repair the tear(s). A repair allows the entire meniscus to be saved and retained whereas removing a peripheral tear would require resection of a very large portion of the meniscus. The key to a successful repair is that the meniscus must be able to heal itself; the repair serves only as a means of securely holding the tissue together long enough for this biologic process to occur.

There are a variety of surgical techniques available for repairing a torn meniscus. Surgeons used to perform an open repair (outside-in), sewing the meniscus back together through an incision. Meniscal repairs are now carried out arthroscopically. One arthroscopic technique is an inside-out method that uses curved cannulas to direct a pair of long needles into the meniscus and out through a small incision in the back of the knee. The suture thread ends connected to the needles are then tied together on the outside of the knee capsule layer to firmly bring the meniscal tear together. This technique works well but note that it does require a relatively large incision to access the site where the knots are tied down.

There are now a variety of methods available to the arthroscopic surgeon that permit a true inside-in repair using minimally invasive surgical techniques. Some of these include bioresorbable T-arrows and dissolving staples which can each be applied from within the joint. T-Fix® sutures have an anchor that acts like a wall anchor and is deployed after placing the suture through the meniscus, the tear and the peripheral rim. Multiple sutures pairs are placed through long hollow needles and are tied together from inside the joint using a knot pusher instrument that compresses the tissue during the repair. Since existing techniques remain time-consuming, the need remains for a more expeditious approach to meniscal repair.

### SUMMARY OF THE INVENTION

This invention relates generally to meniscal repair and, in particular, to instruments and methods directed to expedite minimally invasive surgical procedures. Apparatus according to the preferred embodiment includes a tissue fastener with a female portion and a male portion with a head and a shaft configured to engage with the female portion. A first elongated member is used to hold the female portion of the fastener behind tissue to be repaired. A second elongated member is used to hold the male portion of the fastener in front of tissue to be repaired in alignment with the female portion. The second elongated member is slideable relative to the first elongated member, allowing a user to pierce and compress the tissue as the male portion engages with the female portion.

In the preferred embodiment, the male and female portions of the fastener are constructed of a bioabsorbable material such as polylactic acid. The shaft of the male fastener is preferably ribbed or barbed, and the female portion of the fastener includes an aperture with features that engage with the ribs or barbs of the male portion, thereby facilitating a desired degree of compression as the portions engage. The head of the male fastener may also be angled for conformance with the outer surface of the tissue undergoing the repair.

The male portion may have an integral pointed tip, or such a tip may be provided through a separate introducer. In alternative embodiments, the male portion of the fastener includes a plurality of shafts and the female portion includes a corresponding set of receiving apertures. According tot yet a further alternative, the male portion includes a length of suture extending therefrom. As a further alternative, a length of suture material may connect multiple fasteners, thereby providing a knotless repair as the fasteners are advanced.

A minimally invasive method of tissue repair according to the invention includes the steps of: loading the fastener portions onto the respective elongated members; locating the female fastener behind a region of tissue to be repaired; sliding the second elongated member such that the male portion of the fastener pierces the tissue and engages with the female portion, thereby compressing the tissue; and repeating these steps as necessary to achieve a desired level of tissue repair.

### BRIEF DESCRIPTION OF THE DRAWINGS

FIG. 1A is a drawing of a knee joint which has been separated to show healthy meniscus tissue;

FIG. 1B is a drawing of a separated knee joint which illustrates a tear in the medial posterior meniscus;

FIG. 2A is a side-view drawing of a meniscus repair instrument according to the invention prior to fastener loading;

FIG. 2B is a drawing of the preferred embodiment, with fasteners loaded;

FIG. 2C is a drawing of the preferred embodiment approaching a meniscal tear;

FIG. 2D is a drawing showing the tear being compressed and repaired by virtue of the invention;

FIG. 2E is a drawing which shows the instrument removed and the fastener remaining in place;

FIG. 3A is an oblique perspective view drawing showing the way in which the preferred embodiment is inserted with respect to a meniscal tear; and

FIG. 3B is an oblique perspective drawing showing multiple fasteners placed across a tear according to the invention;

FIG. 4 is a drawing of a multi-prong fastener according to the invention;

FIG. 5 is a drawing of a fastener including a length of suture;

FIG. 6 is a drawing that shows how a length of suture may be provided between two fasteners;

FIG. 7 is a drawing that shows an introducer for a vertical multi-prong fastener;

FIG. 8 is a drawing that shows an introducer for a horizontal multi-prong fastener;

FIG. 9 shows the use of a sharpened tip in conjunction with a hollow fastener;

FIG. 10 shows the alternate use of a split-ring or tubular female portion; and

FIG. 11 illustrates an alternate head shape.

### DETAILED DESCRIPTION OF THE INVENTION

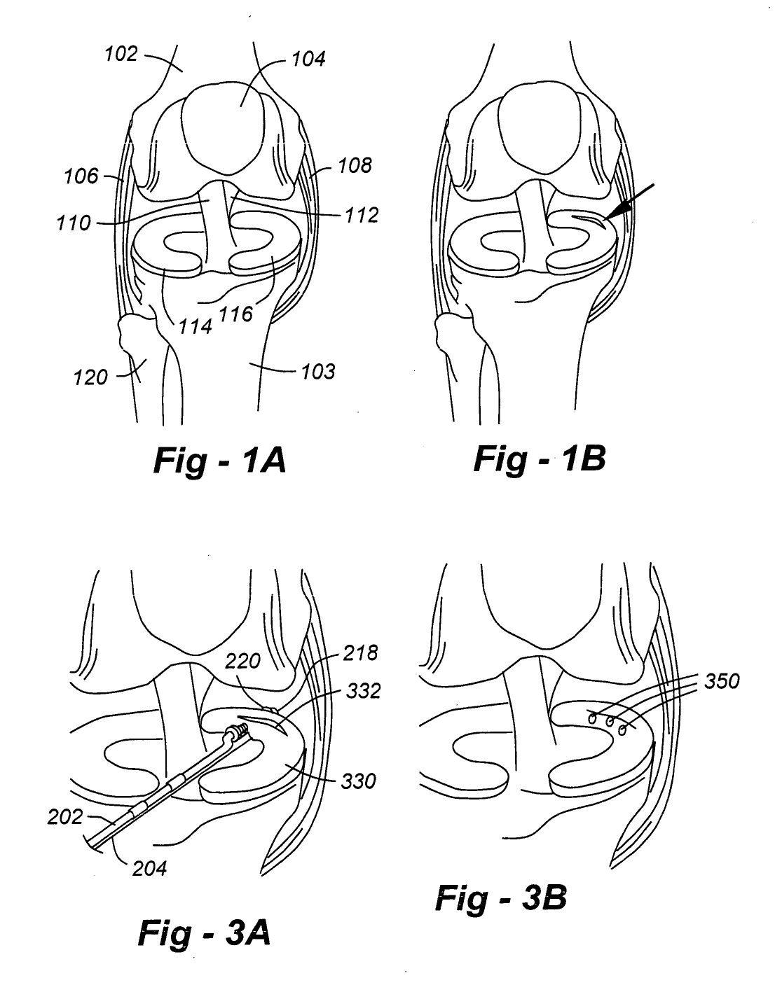

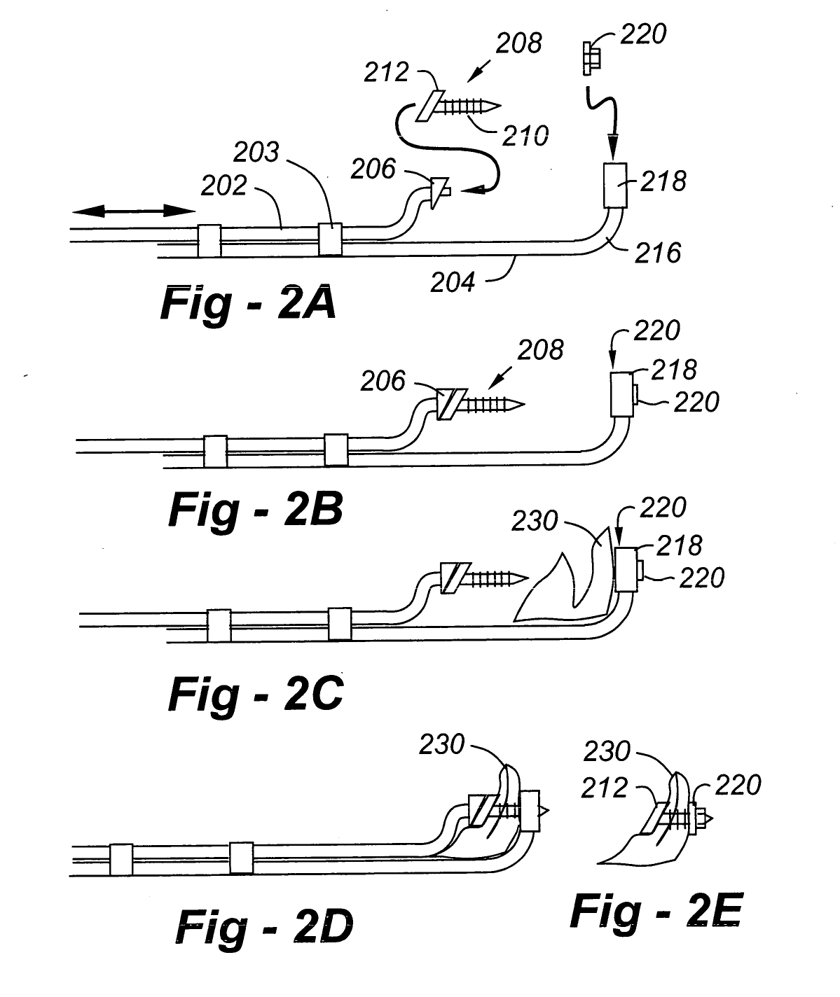

Turning now to the drawings, and having discussed FIGS. 1A and 1B, FIG. 2A presents a side-view drawing of an instrument according to the invention used for the repair of meniscus tissue. The instrument includes a lower arm 204, bent at 216, and terminating in a holder 218 for the female portion 220 of a fastener according to the invention. An upper arm 202, which is slideable relative to the lower arm 204, is physically configured to provide a holder 206 for the male portion 208 of the fastener. The arms may further include markings to indicate when engagement has occurred.

In the preferred embodiment, the fastener portions 208, 220, are preferably constructed of a bioabsorbable material, such as polylactic acid. The male portion of the fastener 208 includes a head 212 and a serrated shaft 120 terminating in a pointed end. The serrations may take the form of ribs, barbs, or other types of spaced-apart features. The female portion is washer-like, and includes one or more corresponding ribs, barbs, or other types of spaced-apart features. In the preferred embodiment, the male fastener is generally cylindrical and the female fastener is generally circular; however, the male portion may be flattened, or constructed with a rectangular cross section with a similarly shaped female fastener. Both portions may be of varying length; for example, the female portion may be more tube-like or alternatively, may be a ‘split ring,’ as shown in FIG. 8.

Continuing the reference to FIG. 2, when the male portion 208 is inserted into the female portion 220, it locks into place with a desired level of compression. The head 212 of the male portion may be slanted, as shown, to conform with the angle of the meniscal or other tissue being repaired, thereby reducing complications and irritations following the procedure.

FIG. 2A shows a preferred embodiment of the invention, with the fastener portions not yet installed in respective holders 206, 218, and with upper arm 202 retracted through mounts 203, which facilitate sliding. With the exception of the fastener portions, the instrument is constructed from a sterilizable or disposable sufficiently rigid material for its intended purpose, such as stainless steel, or the like.

FIG. 2B is a side-view drawing of the preferred embodiment with the fastener portions mounted in respective holders 206, 218. FIG. 2C shows the instrument inserted under meniscal tissue 230, such that holder 218, including the distal portion of the fastener, is behind a tear to be repaired. Those of skill will recognize that other approaches, including approaches over the tear, may alternatively be used. In FIG. 2D, the upper arm 202 has been advanced, causing the proximal portion of the fastener to extend through the distal portion of the fastener, and lock into position when a desired degree of compression has been achieved. Once this takes place, the instrument can be removed, leaving the tear compressed and the meniscus tissue restored, as shown in FIG. 2E.

FIG. 3A is a perspective view drawing of a separated knee joint, showing the way in which the instrument with fasteners is placed relative to a tear 332 in a meniscus 330. Usually, the surgeon is able to slip the instrument under the meniscus in the vicinity of the tear, then turn the instrument to an appropriate position to facilitate compression and repair. A such, the instrument may be curved or angled along its length to facilitate placement. After placing one fastener system, the instrument may be withdrawn, reloaded, and reinserted to install multiple fasteners, as appropriate, as shown in FIG. 3b.

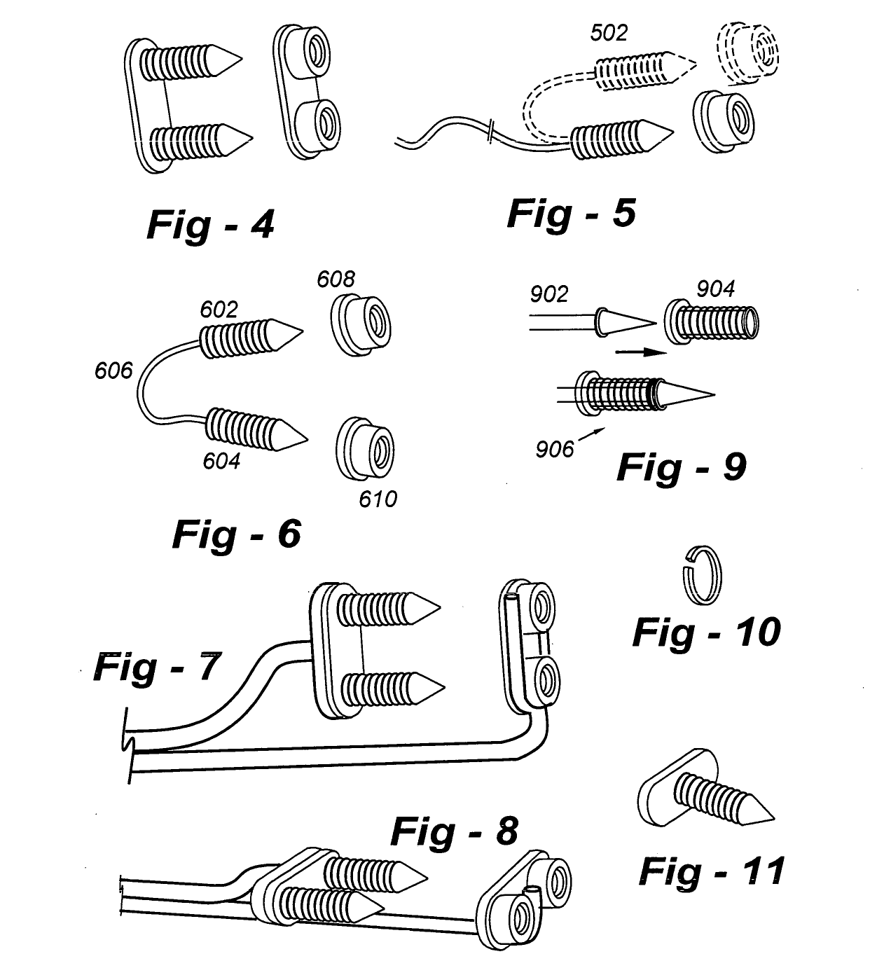

To further expedite the procedure, the two-prong fastener of FIG. 4 may be used, wherein the proximal portion is staple-shaped, and the distal portion is a link member having two apertures to receive the prongs of the staple. Alternatively, two separate distal fasteners may be employed. With this alternative arrangement, fewer fasteners may be used, allowing a smaller tear to be repaired with a single fastener, in some cases. As a further alternative, the male portion may have a suture extending therefrom, as depicted in FIG. 5, allowing knots to be tied following the introduction of multiple fasteners.

FIG. 6 is a drawing that shows how a length of suture 606 may be provided between two fasteners 602, 604, and either separate female portions 608, 610 or coupled female receptacles as shown in FIG. 4. When multiple fasteners are used simultaneously, they may be oriented vertically with the introducer shown in FIG. 7, horizontally using the introducer shown in FIG. 8, or at any angle with appropriate instrumentation. Multiple fasteners may also be loaded into ‘clips,’ allowing them to be introduced in sequence without having to remove the introducer from the knee compartment to reload.

FIG. 9 shows the use of an introducer 902 having a sharpened tip in conjunction with a hollow fastener 904. The assembly, indicated at 906, allows the use a metal tip, for example, which is withdrawn following tissue compression. Using such a hollow device may be advantageous for repair permitting a channel to exist across the repair for blob clots, angioblasts, fibroblasts, and so forth, to bridge the torn segments and facilitate healing. As an alternative to a barrel with ribs or grooves, the female portion may be in the form of a split-ring or longer split tube (not shown). Different head shapes may also be provided, such as the ‘winged’ head shown in FIG. 11.

I claim:

1. Tissue repair apparatus, comprising:
a tissue fastener including a female portion and a male portion with a shaft configured to engage with the female portion;
a first elongated member for holding the female portion of the fastener behind tissue to be repaired;
a second elongated member for holding the male portion of the fastener in front of tissue to be repaired in alignment with the female portion; and
wherein the second elongated member is slideable relative to the first elongated member, allowing a user to pierce the tissue and compress the tissue as the male portion engages with the female portion.

2. The tissue repair apparatus of claim 1, wherein the male and female portions of the fastener are bioabsorbable.

3. The tissue repair apparatus of claim 1, wherein the shaft of the male fastener is ribbed or barbed.

4. The tissue repair apparatus of claim 1, wherein:
the shaft of the male fastener is ribbed or barbed; and
the female portion of the fastener includes a feature that engage with the ribs or barbs of the male portion.

5. The tissue repair apparatus of claim 1, wherein the male fastener includes a head that is perpendicular or angled relative to the axis of the shaft.

6. The tissue repair apparatus of claim 1, wherein the male portion of the fastener includes a plurality of shafts, and the female portion includes a corresponding set of receptacles.

7. The tissue repair apparatus of claim 1, wherein the male portion of the fastener is a staple.

8. The tissue repair apparatus of claim 1, wherein the male portion includes a length of suture extending therefrom.

9. The tissue repair apparatus of claim 1, including two male portions connected to one another with a length of suture material.

10. The tissue repair apparatus of claim 1, wherein the male portion includes an integral sharpened tip.

11. The tissue repair apparatus of claim 1, wherein:
the male portion is substantially hollow; and
the apparatus further includes an introducer having a sharpened tip.

12. An instrument for minimally invasive tissue repair using a fastener having a female portion and male portion, the instrument comprising:
a first elongated member for holding the female portion of the fastener behind tissue to be repaired;
a second elongated member for holding the male portion of the fastener in front of tissue to be repaired in alignment with the female portion; and
wherein the second elongated member is slideable relative to the first elongated member, allowing a user to pierce and compress the tissue as the male portion engages with the female portion.

13. The instrument of claim 12, further including markings to indicate engagement of the male and female portions.

14. A minimally invasive method of tissue repair, comprising the steps of:
a) providing the apparatus of claim 1;
b) locating the female fastener behind a region of tissue to be repaired;
c) sliding the second elongated member such that the male portion of the fastener pierces the tissue and engages with the female portion, thereby compressing the tissue; and
repeating steps b) and c) as necessary to achieve a desired level of tissue repair.

15. The method of claim 14, wherein the tissue is meniscus tissue.

16. Tissue repair apparatus, comprising:
a tissue fastener including a female portion and a hollow male portion configured to engage with the females portion;
a first elongated member to position the female portion behind tissue to be repaired;
a second elongated member for holding the male portion of the fastener in front of tissue to be repaired in alignment with the female portion; and
wherein the second elongated member is slideable relative to the first elongated member, allowing a user to pierce the tissue and compress the tissue as the male portion engages with the female portion.

17. The tissue repair apparatus of claim 16, wherein the male and female portions of the fastener are bioabsorbable.

18. The tissue repair apparatus of claim 16, wherein the shaft of the male fastener is ribbed or barbed.

19. The tissue repair apparatus of claim 16, wherein:
the shaft of the male fastener is ribbed or barbed; and
the female portion of the fastener includes a feature that engage with the ribs or barbs of the male portion.

20. The tissue repair apparatus of claim 16, wherein the male fastener includes a head that is perpendicular or angled relative to the axis of the shaft.

21. The tissue repair apparatus of claim 16, wherein the male portion of the fastener includes a plurality of shafts, and the female portion includes a corresponding set of receptacles.

22. The tissue repair apparatus of claim 16, wherein the male portion of the fastener is a staple.

23. The tissue repair apparatus of claim 16, wherein the male portion includes a length of suture extending therefrom.

24. The tissue repair apparatus of claim 16, including two male portions connected to one another with a length of suture material.

25. The tissue repair apparatus of claim 16, wherein the male portion includes an integral sharpened tip.
